(19) United States
(12) Patent Application Publication
(10) Pub. No.: US 2016/0271126 A1
(43) Pub. Date: Sep. 22, 2016

(54) PHARMACEUTICAL COMPOSITION

(71) Applicant: Hoffmann-La Roche Inc., Little Falls, NJ (US)

(72) Inventors: Stephanie Buurman, Riehen (CH); Axel Schiffmann, Weil am Rhein (DE)

(73) Assignee: Hoffmann-La Roche Inc., Little Falls, NJ (US)

(21) Appl. No.: 15/170,025

(22) Filed: Jun. 1, 2016

Related U.S. Application Data
(63) Continuation of application No. PCT/EP2014/076035, filed on Dec. 1, 2014.

(30) Foreign Application Priority Data
Dec. 3, 2013 (EP) 13195401.8

(51) Int. Cl. A61K 31/496; A61K 9/28; A61K 9/20

(52) U.S. Cl. CPC A61K 31/496; A61K 9/2059; A61K 9/2027; A61K 9/2054; A61K 9/2009; A61K 9/2013; A61K 9/2095; A61K 9/2893; A61K 9/284; A61K 9/2018

(57) ABSTRACT

The invention relates to pharmaceutical composition in form of a film-coating tablet comprising bitopertin or a salt thereof.

### CROSS REFERENCE TO RELATED APPLICATIONS

This application is a continuation of International Application No. PCT/EP2014/076035 having an international filing date of Dec. 1, 2014 and which claims benefit under 35 U.S.C. §119 to European Patent Application No. 13195401.8 filed Dec. 3, 2013. The entire contents of both are incorporated herein by reference.

### FIELD OF THE INVENTION

The invention relates to a pharmaceutical composition for low water soluble drugs and, in particular, to a pharmaceutical composition comprising bitopertin or a salt thereof.

### BACKGROUND OF THE INVENTION

Bitopertin ([4-(3-fluoro-5-trifluoromethyl-pyridin-2-yl)-piperazin-1-yl]-(5-methanesulfonyl-2-(S)-2,2,2-trifluoro-1-methyl-ethoxy)phenyl]-methanone) is described in WO2005/014563, and it can be used for the treatment of schizophrenia and other psychotic disorders by inhibition of the glycine transporter (GlyT1), the main glycine transporter in the forebrain. Thus, if a glutamate deficit is implicated in the pathophysiology of schizophrenia, enhancing glutamate transmission, in particular via NMDA receptor activation, would be predicted to produce both anti-psychotic and cognitive enhancing effects.

Thus, increasing activation of NMDA receptors via GlyT-1 inhibition may lead to agents that treat psychosis, schizophrenia, dementia and other diseases in which cognitive processes are impaired, such as attention deficit disorders or Alzheimer's disease.

Bitopertin is especially useful for the treatment of negative symptoms in schizophrenia, which are apathy, social withdrawal, emotional blunting, impaired ability to anticipate pleasure in everyday life and for the treatment of cognitive impairment such as for the treatment of difficulties with working memory, attention and planning.

### SUMMARY OF THE INVENTION

In one aspect of the present invention there is provided a pharmaceutical tablet composition containing a core comprising bitopertin, or a salt thereof, lactose monohydrate, maize starch, croscarmellose sodium, povidone 30, microcrystalline cellulose, talc, magnesium stearate said core coated with Opadry® II .

In another aspect of the present invention there is provided a method to prepare a pharmaceutical tablet composition containing a core comprising bitopertin, or a salt thereof, lactose monohydrate, maize starch, croscarmellose sodium, povidone 30, microcrystalline cellulose, talc, magnesium stearate said core coated with Opadry® II.

### BRIEF DESCRIPTION OF THE FIGURE

FIG. 1 depicts the extent of dissolution of the bitopertin as a function of time. The five batches included in FIG. 1 are those shown in Table 1.

### DETAILED DESCRIPTION OF THE INVENTION

It has now been found that a pharmaceutical composition comprising bitopertin in a dose of 3, 5, 10, 20, 30 or 60 mg is particularly efficient in treating negative or cognitive symptoms in schizophrenia. Furthermore, the tablet formulation in accordance with the invention may be suitable for the treatment of deficits in social communications and interactions in autism spectrum disorders (ASD), in the treatment of apathy in early Alzheimer's disease (AD), in the treatment of residual symptoms of motivation after a recent major depressive episode, in the treatment of post traumatic stress disorder (PTSD) and for the treatment of apathy and cognitive deficits in Parkinson's disease (PD).

The invention relates also to a pharmaceutical composition comprising 3, 30 and 60 mg bitopertin in a film-coated tablet. It was surprisingly found that a pharmaceutical composition comprising bitopertin in a tablet form with a dose range of 3, 5, 10, 20, 30 and 60 mg is particularly efficient in treating or preventing the above mentioned diseases. The preferred dosage range is 5, 10 and 20 mg of bitopertin.

The present invention provides an easy-to swallow solid dosage form which improves patient compliance. The present tablet formulation effectively delivers the desired dosage, and is sufficiently robust for transport and packaging. The present tablet performance was chosen to meet the criteria for an immediate release formulation and it was aimed at having more than 80% of the drug load dissolved within 15 minutes with a maximum amount liberated after 60 minutes. A film-coating was applied to mask the bitter taste.

Bitopertin is practically insoluble in aqueous buffers and in water, but is freely soluble in polar organic solvents. Being a BCS 2 molecule (according to the Biopharmaceutics Classification System, wherein the molecule has high permeability and low solubility), the API particles size distribution has been identified as a major critical quality attribute of the drug substance. In vivo studies in both humans and cynomolgus monkeys showed a direct correlation between the particle size distribution of the API in tablets and the bioavailability of the formulation. Thus, micronized API was selected for development of a suitable formulation.

More particularly, the invention relates to a pharmaceutical composition comprising 5, 10 or 20 mg bitopertin in a 155 mg film-coated tablet, obtainable by
(i) Mixing bitopertin with lactose monohydrate, maize starch, crosarmellose sodium and povidone,
(ii) Granulating the mixed powder from step i using purified water,
(iii) Drying and screen the granulate from step ii,
(iv) Adding talc, magnesium stearate and microcrystalline cellulose to the granulate from step iii and mix,
(v) Compressing the granulate into tablets,
(vi) Preparing the film-coating suspension using a pre-mixture, containing polyvinylalcohol, titanium dioxide, macrogol 3350, talc and optionally yellow iron oxide and purified water, and
(vii) Film-coating the tablets with the suspension from step vi.

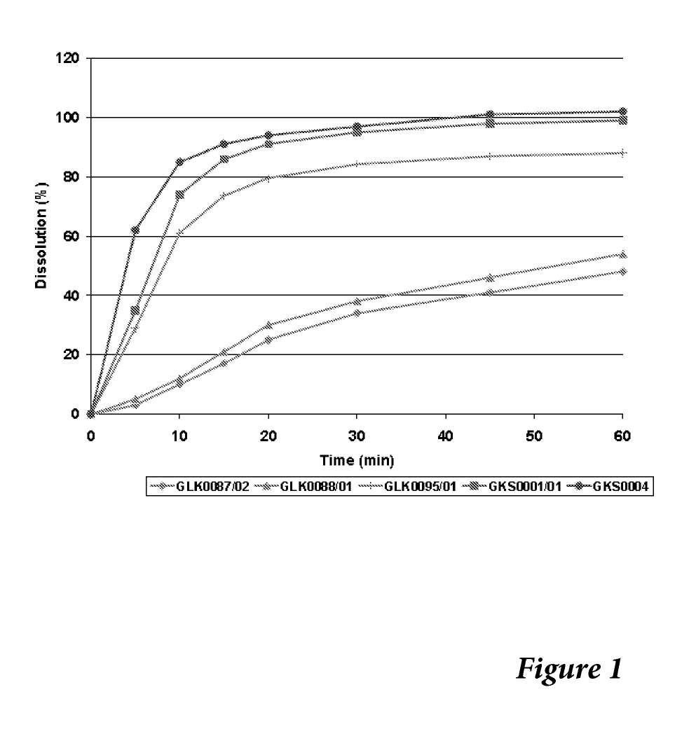

In order to optimize the bioavailability and the dissolution performance, different excipients were tested as fillers, binder, disintegrant and lubricant. It has surprisingly been found that the dissolution behavior of the resulting tablets was dependent on the particle size distribution of the selected excipients. The dissolution was better, the finer the particle of the selected excipients was (see FIG. 1) and a combination of corn starch and lactose provided the finest mean particle size distribution with the best tablet dissolution, which was also able to ensure suitable granulate particle size distribution, for appropriate flow of the final blend for tablet compression. It is hypothesized that micronized bitopertin could be optimally dispersed finely within the tablet formulation only when using filler excipients with a fine mean particle size thus reducing the risk of API sintering during the compaction procedure.

API sintering would result in larger API particles and thus a decrease in dissolution and bioavailability.

TABLE 1
Evaluation of different filler/diluent combinations
for 30 mg bitopertin tablet

| | | d50 | | | d50 | |
|---|---|---|---|---|---|---|
| Filler | % | [μm] | Diluent | % | [μm] | Batch |
| D-Mannit | 30 | 160 | Avicel PH102 | 33.5 | 100 | GLK0087/02 |
| Lactose | 30 | 30 | Avicel PH102 | 33.5 | 100 | GLK0088/01 |
| Corn starch | 30 | 10-25 | Avicel PH102 | 33.5 | 100 | GLK0095/01 |
| Corn starch | 30 | 10-25 | lactose | 33.5 | 30 | GKS0001/01 |
| Corn starch | 30 | 10-25 | Avicel PH101 | 35.5 | 50 | GKS0004 |

In the present description the terms “diluent” and ‘filler’ refer to excipients which fills out the size of a tablet or capsule, making it practical to produce and convenient for the consumer to use. Suitable diluents and fillers include e.g. pharmaceutically acceptable inert fillers, such as microcrystalline cellulose, lactose monohydrate, dibasic calcium phosphate sugar, sugar alcohols, corn starch, sucrose, silicic anhydride, polysaccharides, N-methyl pyrrolidone (Pharmasolve (ISP)) and mixtures thereof. The term sugar and sugar alcohols comprises mannitol, lactose, fructose, sorbitol, xylitol, maltodextrin, dextrates, dextrins, lactitol and mixtures thereof.

Binders are added to tablet formulations to add cohesiveness to powders thereby providing the necessary bonding to form granules which under compaction form a compact mass as tablet. Methyl cellulose, carboxymethylcellulose, hydroxypropylcellulose, hydroxymethyl propylcellulose, polyvinylpyrrolidone, polyvinyl alcohol, povidone 30, starch and starch pregelatinized are suitable examples of binders. Individual binders, or Mixtures of two, three or more binders can be used in the formulation.

The term “disintegrant” refers to an excipient which expands and dissolve when wet causing the tablet to break apart in the digestive tract, and via the increase in surface area available for dissolution facilitation release of the active ingredients for absorption. Suitable disintegrants include e.g. lightly crosslinked polyvinyl pyrrolidone, corn starch, potato starch, maize starch, and modified starches, croscarmellose sodium, carboxymethylcellulose calcium, carboxymethylcellulose sodium, crossprovidone, sodium starch glycolate and mixtures thereof.

Suitable lubricants, including agents that act on the flowability of the powder by reducing interparticle friction and cohesion to be compressed, are colloidal silicon dioxide, such as aerosil, talc, stearic acid, magnesium stearate, calcium stearate, glyceryl behenate, sodium stearyl fumarate and silica gel.

Microcrystalline cellulose is used as a compression aid for the tablet.

The term “coating” refers to an excipient which is applied on the surface of a tablet and which protects tablet ingredients from deterioration by moisture in the air and make large or unpleasant-tasting tablets easier to swallow. Examples of coating agent include PVA (polyvinyl alcohol), HPMC (hydroxypropylmethyl cellulose) and PEG (polyethylene glycol). In the film coating mixture are also included coloring agents, such as titanium dioxide and iron oxide yellow. A suitable plasticizer in the film coat is Macrogol 3350.

In accordance with the present invention, the formulation for bitopertin is intended to be an oval once-daily film-coated tablet with a size as small as possible (10×4.63 mm) with a tablet weight of 150 mg and 5 mg coat weight. All dose strengths have the same size, to be differentiated by engraving and different color shades. The tablet is an oval, biconvex film-coated tablet.

One object of the present invention is a film-coated tablet, comprising 3, 5, 10, 20, 30 and 60 mg bitopertin for 150 mg tablet weight and a coat weight of 5 mg.

A film-coated tablet contains:

### EXAMPLE 1

### 5 mg Bitopertin for 150 mg Tablet Weight (and 5 mg Coat Weight)

| | Weight | |
|---|---|---|
| Component | (mg/tablet) | Function |
| Bitopertin | 5.00 | drug substance |
| Lactose monohydrate | 78.75 | diluent |
| Maize starch | 37.50 | filler |
| Croscarmellose sodium | 3.75 | disintegrant |
| Povidone 30 | 6.25 | binder |
| Microcrystalline cellulose | 15.00 | compression aid |
| Talc | 3.00 | lubricant |
| Magnesium stearate | 0.75 | lubricant |
| Total kernel weight | 150.00 | |
| Opadry II white 85F18422 | 5.00 | film coat |
| (polyvinyl alcohol, | | (coating agent, |
| titanium dioxide, | | coloring agent, |
| macrogol 3350, | | plasticizer, |
| talc) | | lubricant) |
| Total film-coat weight | 5.0 | |
| Total tablet weight | 155.00 | |

### EXAMPLE 2

### 10 mg Bitopertin for 150 mg Tablet Weight

| | Weight | |
|---|---|---|
| Component | (mg/tablet) | Function |
| Bitopertin | 10.00 | drug substance |
| Lactose monohydrate | 73.75 | diluent |
| Maize starch | 37.50 | filler |
| Croscarmellose sodium | 3.75 | disintegrant |
| Povidone 30 | 6.25 | binder |
| Microcrystalline cellulose | 15.00 | compression aid |
| Talc | 3.00 | lubricant |
| Magnesium stearate | 0.75 | lubricant |
| Total kernel weight | 150.00 | |
| Opadry II white 85F18422 | 5.00 | film coat |
| (polyvinyl alcohol, | | (coating agent, |
| titanium dioxide, | | coloring agent, |
| macrogol 3350, | | plasticizer, |
| talc) | | lubricant, |
| iron oxide yellow) | | coloring agent) |
| Total film-coat weight | 5.0 | |
| Total tablet weight | 155.00 | |

### EXAMPLE 3

### 20 mg Bitopertin for 150 mg Tablet Weight

| | Weight | |
|---|---|---|
| Component | (mg/tablet) | Function |
| Bitopertin | 20.00 | drug substance |
| Lactose monohydrate | 63.75 | diluent |
| Maize starch | 37.50 | filler |
| Croscarmellose sodium | 3.75 | disintegrant |
| Povidone 30 | 6.25 | binder |
| Microcrystalline cellulose | 15.00 | compression aid |
| Talc | 3.00 | lubricant |
| Magnesium stearate | 0.75 | lubricant |
| Total kernel weight | 150.00 | |
| Opadry II white 85F18422 | 5.00 | Film coat |
| (polyvinyl alcohol, | | (coating agent, |
| titanium dioxide, | | coloring agent, |
| macrogol 3350, | | plasticizer, |
| talc, | | lubricant, |
| iron oxide yellow) | | coloring agent) |
| Total film-coat weight | 5.0 | |
| Total tablet weight | 155.00 | |

Tablets containing 3, 30 and 60 mg bitopertin contain 83.75 mg of bitopertin and lactose monohydrate combined in the tablet. Thus a tablet with 3 mg bitopertin contains 80.75 mg lactose monohydrate, or a tablet with 30 mg bitopertin contains 53.75 mg lactose monohydrate, or a tablet with 60 mg bitopertin contains 23.75 mg lactose monohydrate.

The invention also relates to a pharmaceutical composition as described above for use as medicament, and preferably for use as medicament for the treatment of schizophrenia and other psychotic disorders, especially for the treatment of negative or cognitive symptoms in schizophrenia, for the treatment of deficits in social communications and interactions in autism spectrum disorders (ASD), in the treatment of apathy in early Alzheimer's disease (AD), in the treatment of residual symptoms of motivation after a recent major depressive episode, in the treatment of post traumatic stress disorder (PTSD) and for the treatment of apathy and cognitive deficits in Parkinson's-disease (PD).

Furthermore, the invention also relates to the use of a pharmaceutical composition as described above for the treatment of the above-mentioned diseases.

The invention further relates to a method of treatment of schizophrenia and other psychotic disorders, especially for the treatment of negative or cognitive symptoms in schizophrenia, for the treatment of deficits in social communications and interactions in autism spectrum disorders (ASD), in the treatment of apathy in early Alzheimer's disease (AD), in the treatment of residual symptoms of motivation after a recent major depressive episode, in the treatment of post traumatic stress disorder (PTSD) and for the treatment of apathy and cognitive deficits in Parkinson's-disease (PD).comprising the step of administering a pharmaceutical composition as described above to a patient in need thereof.

More particularly, the invention relates to a pharmaceutical composition comprising 3, 5, 10, 20 30 or 60 mg bitopertin in a 150 mg tablet size with 5 mg coat weight, obtainable by
(i) Mixing bitopertin with a diluent, filler, desintegrant and binder,
(ii) Granulating the mixed powder from step (i) using purified water,
(iii) Drying and screen the granulate from step (ii),
(iv) Adding lubricants and compression aid to the granulate from step (iii) and mix,
(v) Compressing the granulate into tablets,
(vi) Preparing the film-coating suspension using a pre-mixture, containing a coating agent, coloring agents, plasticizer, lubricant and purified water, and
(vii) Film-coating the tablets with the suspension from step (vi).
   More specifically, the invention relates to a pharmaceutical composition comprising 3, 5, 10, 20, 30 or 60 mg bitopertin in a 150 mg tablet size with 5 mg coat weight, obtainable by
(i) Mixing bitopertin with lactose monohydrate, maize starch, crosarmellose sodium and povidone,
(ii) Granulating the mixed powder from step (i) using purified water,
(iii) Drying and screen the granulate from step (ii),
(iv) Adding talc, magnesium stearate and microcrystalline cellulose to the granulate from step (iii) and mix,
(v) Compressing the granulate into tablets,
(vi) Preparing the film-coating suspension using a pre-mixture, containing polyvinylalcohol, titanium dioxide, macrogol 3350, talc and yellow iron oxide and purified water, and
(vii) Film-coating the tablets with the suspension from step vi.
   For the preparation of the tablet, the following procedure can be followed:
   The tablets are manufactured using the conventional pharmaceutical operations of wet-high shear granulation, fluid bed drying, blending, compression, and film coating.
   Detailed description of the manufacturing process:
1. Load bitopertin milled drug substance, lactose monohydrate, maize starch, croscarmellose sodium and Povidone 30 in a high shear mixer.
2. Blend for 5 to 10 min at suitable rpm.
3. Add water in the course of 10 min. and knead for further 5 to 10 min at suitable rpm.
4. The wet granules are transferred into the fluid bed dryer over a 5 mm sieve and dried under the following conditions: inlet air temperature: 50 to 70° C., air flow: 300 to 600 m³/h. Drying end point 4% (range: 2.5-5.5%) determined by LOD, Duration: 45 min.
5. Screen the granules through a 1.5 mm Frewitt cone sieve at suitable rotor speed.
6. Add microcrystalline cellulose as is and add talc and magnesium stearate through a hand sieve of 0.5 mm to the granules and blend with bin blender for 20 min at 6 rpm.
7. Compress the final blend from [step 6] into tablet core at suitable compression force and rotor speed to obtain tablets with hardness of 50-90N.
8. Prepare the coating suspension using purified water and the Opadry II Yellow 85F32645 film-coating mixture (Polyvinyl Alcohol partially hydrolyzed, titanium dioxide, Macrogol/PEG 3350, talc) and Iron oxide yellow) and stir for at least one hour.
9. Spray the coating suspension from [step 8] onto the tablet cores from [step 7].

We claim:

1. A pharmaceutical tablet composition in form of a core comprising bitopertin, or a salt thereof, and a film coating surrounding said core.

2. The composition according to claim 1 wherein the core comprises bitopertin, or a salt thereof, lactose monohydrate, maize starch, croscarmellose sodium, povidone 30, microcrystalline cellulose, talc, magnesium stearate and said core is coated with Opadry® II.

3. The composition according to claim 2 comprising:

| | Weight |
|---|---|
| Component | (mg/tablet) |
| Bitopertin | 5.00 |
| Lactose monohydrate | 78.75 |
| Maize starch | 37.50 |
| Croscarmellose sodium | 3.75 |
| Povidone 30 | 6.25 |
| Microcrystalline cellulose | 15.00 |
| Talc | 3.00 |
| Magnesium stearate | 0.75 |
| Opadry II white 85F18422 | 5.00. |
| (PVA, TiO₂, macrogol 3350, | |
| talc) | |

4. The composition according to claim 2 comprising:

| | Weight |
|---|---|
| Component | (mg/tablet) |
| Bitopertin | 10.00 |
| Lactose monohydrate | 73.75 |
| Maize starch | 37.50 |
| Croscarmellose sodium | 3.75 |
| Povidone 30 | 6.25 |
| Microcrystalline cellulose | 15.00 |
| Talc | 3.00 |
| Magnesium stearate | 0.75 |
| Opadry II white 85F18422 | 5.00. |
| (PVA, TiO₂, macrogol 3350, | |
| talc, iron oxide yellow) | |

5. The composition according to claim 2 comprising:

| | Weight |
|---|---|
| Component | (mg/tablet) |
| Bitopertin | 20.00 |
| Lactose monohydrate | 63.75 |
| Maize starch | 37.50 |
| Croscarmellose sodium | 3.75 |
| Povidone 30 | 6.25 |
| Microcrystalline cellulose | 15.00 |
| Talc | 3.00 |
| Magnesium stearate | 0.75 |
| Opadry II white 85F18422 | 5.00. |
| (PVA, TiO₂, macrogol 3350, | |
| talc, iron oxide yellow) | |

6. A process for preparing a pharmaceutical table composition according to claim 2 containing 3, 5, 10, 20, 30 or 60 mg of bitopertin, or a salt thereof, comprising the steps of:
(i) Mixing bitopertin with a diluent, filler, desintegrant and binder;
(ii) Granulating the mixed powder from step (i) using purified water;
(iii) Drying and screen the granulate from step (ii);
(iv) Adding lubricants and compression aid to the granulate from step (iii) and mix;
(v) Compressing the granulate from step (iv) into tablets;
(vi) Preparing the film-coating suspension using a pre-mixture, containing a coating agent, coloring agents, plasticizer, lubricant and purified water; and,
(vii) Film-coating the tablets with the suspension from step (vi).

7. The process according to claim 6 comprising the steps of:
(i) Mixing bitopertin with lactose monohydrate, maize starch, croscarmellose sodium and providone;
(ii) Granulating the mixed powder from step (i) using purified water;
(iii) Drying and screening the granulate from step (ii);
(iv) Adding talc, magnesium stearate and microcrystalline cellulose to the granulate from step (iii) and mixing;
(v) Compressing the granulate from step (iv) into tablets;
(vi) Preparing the film-coating suspension using a pre-mixture, containing polyvinylalcohol, titanium dioxide, macrogol 3350, talc and yellow iron oxide and purified water; and,
(vii) Film-coating the tablets with the suspension from step (vi).

8. A method for the treatment of negative or cognitive symptoms in schizophrenia, deficits in social communications and interactions in autism spectrum disorders (ASD), apathy in early Alzheimer's disease (AD), residual symptoms of motivation after a recent major depressive episode, post traumatic stress disorder (PTSD), and for the treatment of apathy and cognitive deficits in Parkinson's-disease, comprising administering to a patient in need thereof a therapeutically effective amount of a pharmaceutical composition according to claim 2.
